SEQUENCE LISTING (26 sequences)

SEQ ID NO: 1 - DNA, 29 bp, Artificial sequence
  Oligonucleotide
gtggggggacatcaagcagccatgcaaat

SEQ ID NO: 2 - DNA, 30 bp, Artificial sequence
  Oligonucleotide
cctttggtccttgtcttatgtccagaatgc

SEQ ID NO: 3 - DNA, 20 bp, Artificial sequence
  GAPGH forward
aacagcctcaagatcagcaa

SEQ ID NO: 4 - DNA, 19 bp, Artificial sequence
  GAPDH forward
ctctcggtgtcggaatgtc

SEQ ID NO: 5 - DNA, 21 bp, Artificial sequence
  GAPDH forward
acgagacccataccaacatcc

SEQ ID NO: 6 - DNA, 19 bp, Artificial sequence
  GAPDH forward
gacctcagccacaaccaac

SEQ ID NO: 7 - DNA, 20 bp, Artificial sequence
  GAPDH forward
ctacaaccgcatcgtcaaac

SEQ ID NO: 8 - DNA, 19 bp, Artificial sequence
  GAPDH forward
actttggatttccccagga

SEQ ID NO: 9 - DNA, 22 bp, Artificial sequence
  GAPDH forward
atctagcactggctggaatgag

SEQ ID NO: 10 - DNA, 21 bp, Artificial sequence
  GAPDH forward
gggttctcttggctgttactg

SEQ ID NO: 11 - DNA, 24 bp, Artificial sequence
  GAPDH forward
gctggaggactttaagggttacct

SEQ ID NO: 12 - DNA, 20 bp, Artificial sequence
  GAPDH forward
tggagtgccaggaggacagt

SEQ ID NO: 13 - DNA, 24 bp, Artificial sequence
  GAPDH forward
ccatccagtgctacttgtgtttac

SEQ ID NO: 14 - DNA, 18 bp, Artificial sequence
  GAPDH forward
gacgcatgccctcaatcc

SEQ ID NO: 15 - DNA, 19 bp, Artificial sequence
  GAPDH forward
cagtctgggtggcagtgat

SEQ ID NO: 16 - DNA, 19 bp, Artificial sequence
  GAPDH forward
aggggggattgaagttctc

SEQ ID NO: 17 - DNA, 21 bp, Artificial sequence
  GAPDH forward
ttcccagacccaatccttatc

SEQ ID NO: 18 - DNA, 20 bp, Artificial sequence
  GAPDH forward
taacccaccagacaaaccac

SEQ ID NO: 19 - DNA, 21 bp, Artificial sequence
  GAPDH forward
cattcagccaggagagagaac

SEQ ID NO: 20 - DNA, 19 bp, Artificial sequence
  GAPDH forward
caggcacaagggctgtatt

SEQ ID NO: 21 - DNA, 23 bp, Artificial sequence
  GAPDH forward
ttcggaggtaacctgtaagtctg

SEQ ID NO: 22 - DNA, 20 bp, Artificial sequence
  GAPDH forward
gcatctgactcctttttcgc

SEQ ID NO: 23 - DNA, 23 bp, Artificial sequence
  GAPDH forward
cttgatgtctgggtcttggttct

SEQ ID NO: 24 - DNA, 20 bp, Artificial sequence
  GAPDH forward
tcttgggtgggtcaggtttg

SEQ ID NO: 25 - DNA, 23 bp, Artificial sequence
  GAPDH forward
ccagttggcttctgttttaggaa

SEQ ID NO: 26 - DNA, 21 bp, Artificial sequence
  GAPDH forward
ctagagcgcaatggtgcaatc

(19) United States
(12) Patent Application Publication
(10) Pub. No.: US 2010/0093655 A1
(43) Pub. Date: Apr. 15, 2010

(54) USE OF RILUZOLE AND DERIVATIVES THEREOF FOR PRODUCING NEW DRUGS

(76) Inventor: Ammar Achour, Creteil (FR)

Correspondence Address: NIXON & VANDERHYE, PC, 901 NORTH GLEBE ROAD, 11TH FLOOR, ARLINGTON, VA, 22203, US

(21) Appl. No.: 12/448,743

(22) PCT Filed: Dec. 28, 2007
(86) PCT No.: PCT/FR2007/002186

§ 371 Date: Jul. 2, 2009

(30) Foreign Application Priority Data
Jan. 3, 2007 (FR) FR 07 000 18

(51) Int. Cl. A61K 31/428; A61K 31/708; A61K 31/7068; A61K 31/513; A61K 31/7072

(52) U.S. Cl. 514/45; 514/367; 514/49; 514/274; 514/50

(57) ABSTRACT

The present invention relates to the use of riluzole or pharmaceutically acceptable salts of riluzole in the production of drugs for restoring and amplifying the immune function in the treatment of infectious diseases and cancers.

The subject of the present invention is the use of riluzole or derivatives thereof for producing new medicaments for restoring and regulating disturbed immune functions in patients suffering from pathological conditions associated with such dysfunctions, such as infectious pathological conditions or cancers.

Riluzole, or 6-(trifluoromethoxy)-2-aminobenzothiazole corresponds to formula (A)

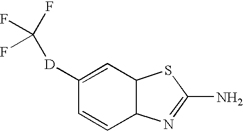

Therapeutic activities have already been reported for riluzole and salts of this compound, for example, activities such as anticonvulsives, anxyolytics and hypnotics (EP 050551).

Application WO 94/20103 also describes their use in the treatment of neuroAIDS, dementia disorders, cognitive disorders, neuropathies, myopathy, ocular disorders and all the neurological symptoms associated with the HIV-1 virus.

The studies by the inventors on these compounds have made it possible to demonstrate that, surprisingly, riluzole, and also derivatives of this product, are capable of restoring the immune function in patients suffering from pathological conditions associated with disregulation of this function. As shown by the examples given in the application, these compounds (this term, as used in the description and the claims, encompasses riluzole and derivatives thereof) are capable of inducing the proliferation of lymphocytes and/or of ensuring their survival in patients suffering from such pathologies, and of inhibiting infection-induced cell apoptosis.

Riluzole is also capable of inducing the expression of interferon α/β and of interleukin 15, for treating a dysfunction associated with a pathological condition of exogenous or endogenous origin.

This is more particularly a pathological condition associated with an attack of iatrogenic origin, induced by the action of immunosuppressants such as cyclosporine or by the action of corticosteroids.

The objective of the invention is therefore to provide a novel use of riluzole or of pharmaceutically acceptable salts thereof, in the production of medicaments for restoring the immune function in the treatment of infectious diseases or cancers.

As pharmaceutically acceptable salts, mention will be made of the salts with inorganic acids, such as hydrochloride, sulfate, nitrate or phosphate or organic acids such as acetate, propionate, succinate, citrate, oxalate, benzoate, fumarate, maleate, methanesulfonate, isethionate, theophillineacetate, salicylate, phenolphthalinate or methylenebis-β-oxynaphthoate, or substitution derivatives of these acids.

The medicaments produced in accordance with the invention may be used in vivo or ex vivo, or else, in the context of tests, in vitro.

They are most generally provided in forms for oral, parenteral, for example intravenous, or topical administration, or else rectal administration, in particular in the form of suppositories.

As solid compositions for oral administration, these medicaments may be used in the form of tablets, pills, powders (gelatin capsules, cachets), or granules. In these compositions, the active ingredient according to the invention is mixed with one or more inert diluents such as starch, cellulose, sucrose, lactose or silica, under an argon stream. These compositions may also comprise substances other than the diluents, for example, one or more lubricants such as magnesium stearate or talc, a coloring, a coating (sugar-coated tablets), or a glaze.

Compositions in liquid form include solutions, suspensions, emulsions, syrups and elixirs, which are pharmaceutically acceptable, containing inert diluents such as water, ethanol, glycerol, plant oils or liquid paraffin. These compositions may comprise substances other than the diluents, for example, wetting products, sweeteners, thickeners, flavorings or stabilizers.

These compositions used in the form of drops or of nebulized products.

The sterile compositions for parenteral administration may be preferably aqueous or non-aqueous solutions, suspensions or emulsions. As solvent or carrier, mention will be made of water, propylene glycol, polyethylene glycol, plant oils, in particular olive oil, injectable organic esters, for example, ethyl oleate, or other suitable organic solvents. These compositions may also contain adjuvants, in particular wetting agents, isotonic agents, emulsifiers, dispersants and stabilizers. The sterilization may be carried out in several ways, for example by asepticizing filtration, by incorporating sterilizing agents into the composition, by irradiation or by heating. They may also be prepared in the form of sterile solid compositions which can be dissolved at the time of use in sterile water or any other injectable sterile medium.

For topical administration, the medicaments advantageously will be in the form of ointments, creams, gels or patches.

The above medicaments advantageously contain, as active ingredient, from 25 to 100 mg of riluzole or of a pharmaceutically acceptable salt of this derivative, these compounds being used alone or in combination, in a given treatment, with other medicaments.

The use of riluzole or of salts thereof, as defined above, makes it possible to have medicaments that are of great value in the treatment of conditions resulting in or responsible for dysfunctions leading to cell death or associated with such disregulation, and also for any pathological situation caused by medicaments, cancers or radiation, or physiological situations, such as old age, and in general, all situations in which the survival and the function of immune cells are impaired or need to be restored.

Mention will in particular by made of the treatment
of pathological conditions associated with a viral attack, in particular the treatment of viral or retroviral infections or of the virological and immune symptoms thereof, induced, for example, by HIV type 1 or 2, including opportunistic infections and infections involving lymphocyte function and immune system regulation, or else infections with the hepatitis A virus, the hepatitis B virus or the hepatitis C virus, or with herpes viruses, or HTLV I or II viruses,
of a parasitic attack, such as leishmaniasis, malaria or schistosomiasis,
of ischemia/reperfusion and of free radical production,
of an attack of iatrogenic origin, for example, induced by the action of immunosuppressants such as cyclosporine or by the action of corticosteroids,
of an attack of tumoral origin, such as a hematological tumor, for instance myeloma, or a tissue tumor,
of pathological conditions associated with an attack which is an autoimmune disease, such as diabetes or thyroiditis.

The medicaments of the invention are also of great advantage in the case of allogenic liver transplants.

Advantageously, the medicaments produced in accordance with the invention are capable of inducing the production of interferons α/β and of IL-15.

The doses used in these treatments depend on the desired effect, on the duration of the treatment and on the route of administration used. They will generally be between 50 and 200 mg per day orally, for an adult, with unit doses ranging from 25 to 100 mg of active substance.

In general, the dosage will be determined according to age and weight and to all the other factors specific to the individual to be treated.

The clinical forms of this composition comprise, in practice, per unit dose or multiple of unit doses, an amount of active substance or mixture of active substances corresponding to a concentration of active substance or mixture of active substances of approximately 1 to 100 nM for an in vitro test.

Those skilled in the art will adjust these amounts case-by-case and/or according to the conditions or physiological states for which an increase in or a tendency toward restoration of cell proliferation and function is desired.

Depending on the route of administration used, the condition of the patient and the active substance used, these doses may vary over time and may be administered according to a dosage comprising administration once or several times per day, per week or per month. Thus, a daily dose as mentioned above can itself be replaced with any other active-substance administration, timing and/or dose (weekly or monthly administration, in particular), insofar as the clinical form of the active ingredient thus administered provides a pharmacological effect substantially similar to that obtained with a daily administration.

The active compositions can be administered together with a carrier or vector or a diluent which is physiologically acceptable, so as to form a complete ready-to-use pharmacological composition.

In the case of HIV infection, studies have reported that infected CD4+ T lymphocytes are capable of restarting viral machinery that was merely dormant. The presence of the provirus in CD4+ memory lymphocytes has thus been detected. These cells, after having actively replicated the virus, are found in the latent state. The same observation has been made in patients treated by tritherapy and who have had no detectable plasma virus for two to thirty months. Whatever the duration of their treatment, they all exhibit reactivatable viruses in their quiescent lymphocytes. These viruses have in fact remained quiescent, evidence of the existence of virus reservoirs through the persistence of provirus in populations of infected lymphocytes, but without any replicative activity.

This reservoir is built up very early on during the infection, probably in the initial phase of the primary infection, and its size remains stable over the course of the infection. It is now known that early treatment of the primary infection does not prevent the formation of the reservoir, but perhaps reduces its size. Since the size of the reservoir overall remains stable, it is probably continuously re-supplied by a low rate of viral replication. It has been shown that active viral replication continues in the reservoir cells over time. Chronic activation of lymphocytes and also lymphocyte cell death are therefore two essential components of AIDS. In fact, T cell activation is an aggravating factor in the progression of the disease, thus promoting viral replication. Thus, the combined action of excessive mortality of hyperstimulated but uninfected T cells and of deficient CD4-lymphocyte regeneration thus can apparently explain the quantitative deficiency in CD4+ T lymphocytes.

The advantage of the medicaments produced in accordance with the invention, which constitute a new treatment approach, will then be measured.

The activity of riluzole in AIDS has been demonstrated in the test for measuring cell survival, apoptosis, typing of lymphocytes and viral production of lymphocytes from AIDS patients, cultured in vitro.

As indicated above, the medicaments defined above are used alone or in combination with other active ingredients for a given treatment.

For the treatment of a pathological condition associated with a viral (or retroviral) attack, use will advantageously be made of a combination with one or more compounds having antiviral properties. In the case of the treatment of AIDS, they may, for example, be compounds with antiviral properties, such as DDI, DDC, antiproteases, 3TC and AZT, or else interferon α, interferon α PEG or ribavirin.

The medicaments of the invention will advantageously be used in alternation with the antivirals in order to enable a pause, due to their toxicity, or else in their presence in order to eradicate the infection.

For the treatment of a pathological condition associated with a viral attack characteristic of herpes, the treatment with the medicaments of the invention will advantageously be combined with an anti-viral compound, for instance acyclovir.

For the treatment of cancers, advantage will advantageously be taken of the induction of the expression of interferon α/β having an immunomodulatory capacity and the capacity to inhibit tumor cell proliferation.

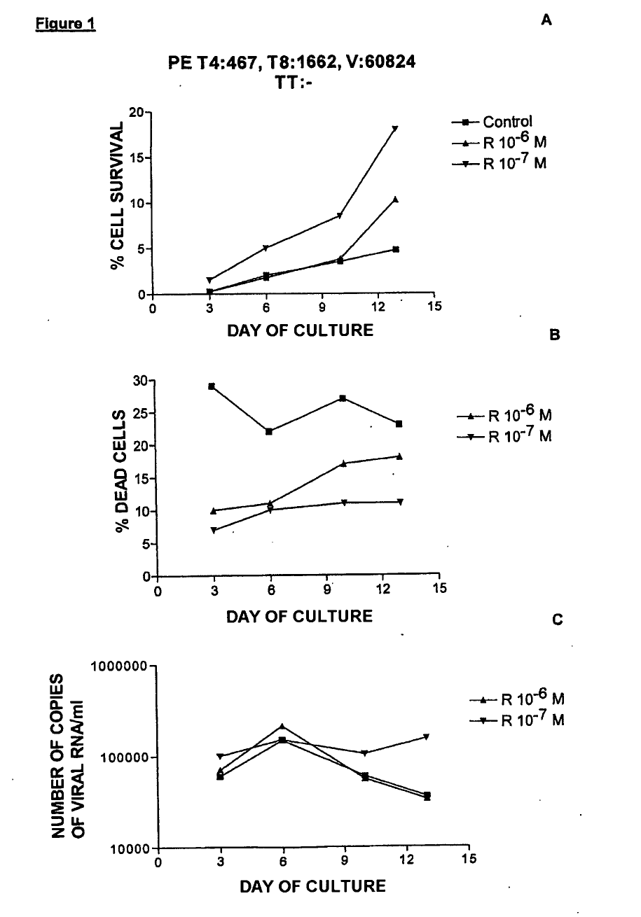

The invention is described in greater detail in the examples which follow, which are given by way of illustration without limiting the scope thereof. In these examples reference is made to FIGS. 1 to 9, which represent, respectively, the effect of riluzole.

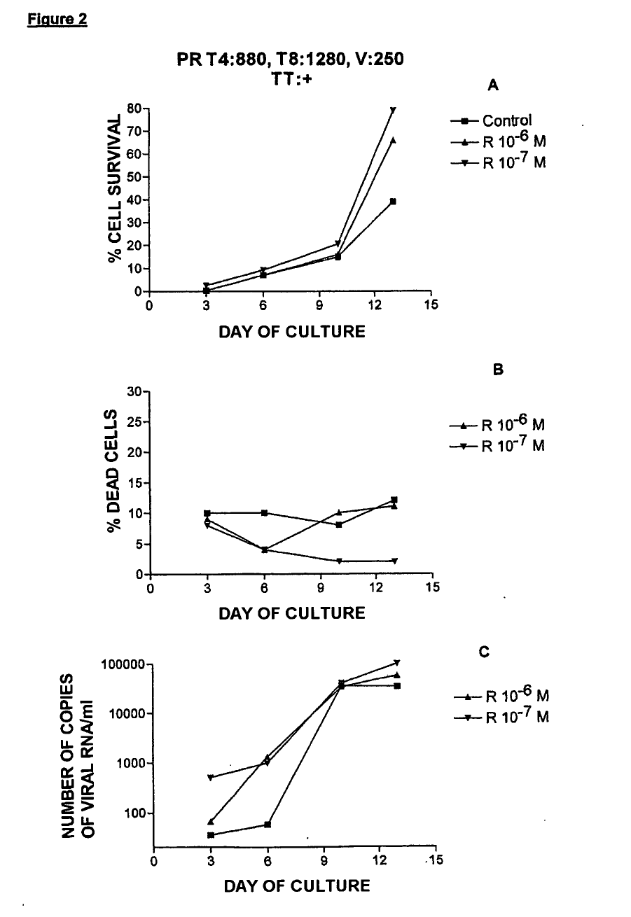

FIGS. 1A to 1C and FIGS. 2A to 2C, on the lymphocytes of patents suffering from AIDS;

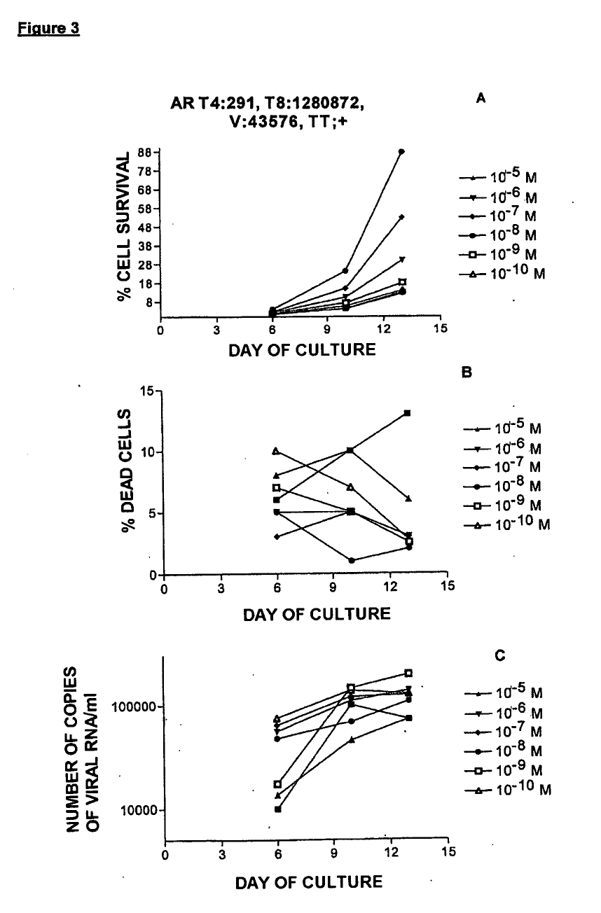

FIGS. 3A to 3C, on the lymphocytes of a patient suffering from AIDS;

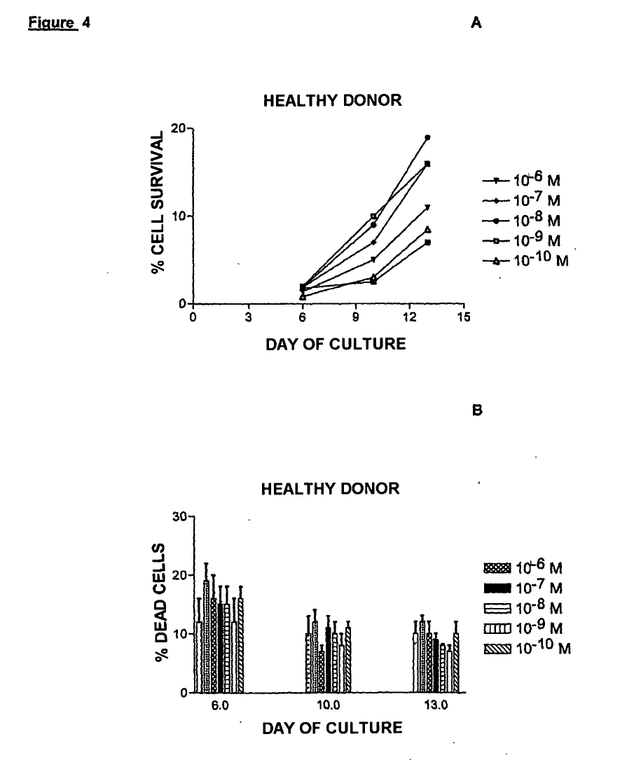

FIGS. 4A and 4B, on the lymphocytes of a healthy seronegative individual;

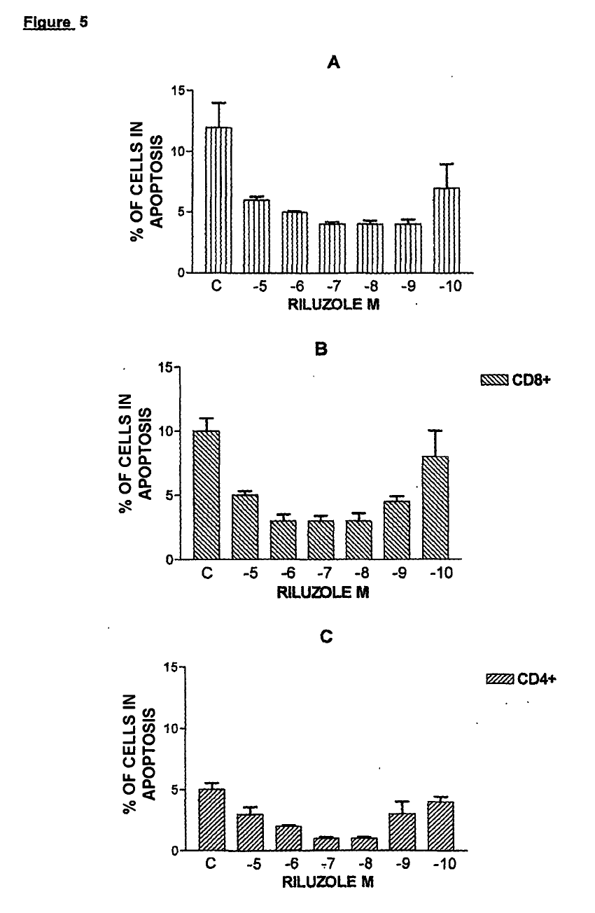

FIGS. 5A and 5B on the total apoptosis expressed by all the lymphocytes, the CD8 lymphocytes and the CD4 lymphocytes;

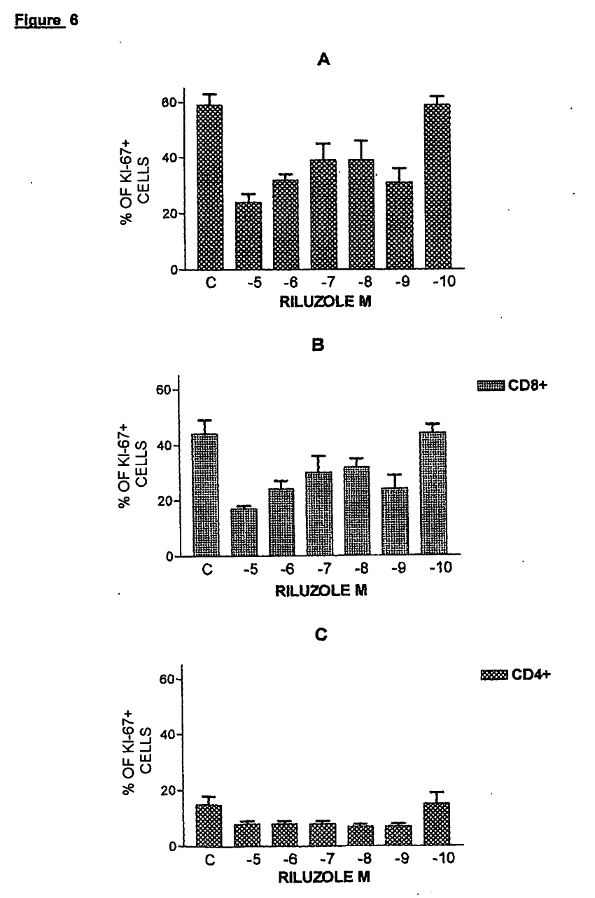

FIG. 6, on the expression of the Ki-67 antigen;

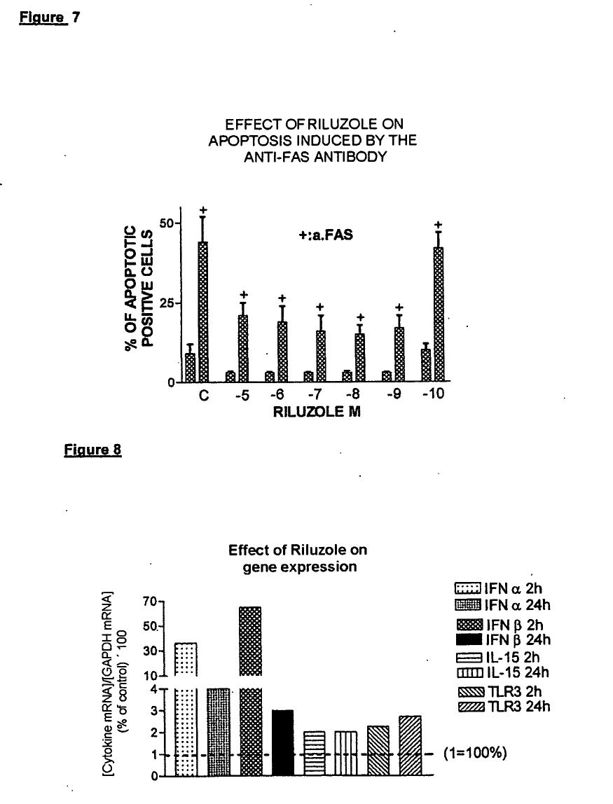

FIG. 7, on the apoptosis induced by the anti-FAS antibody;

FIG. 8, on the expression of cytokine genes; and

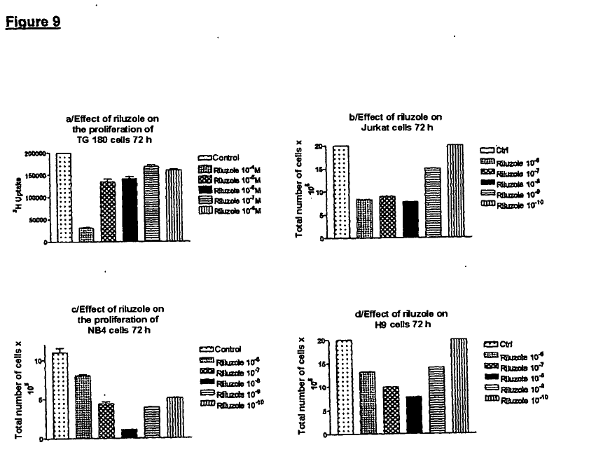

FIG. 9, on tumor cell proliferation.

Unless otherwise mentioned, the dosages of the active ingredient are related back to the volume, in liters, of the blood plasma of the patient.

### EXAMPLE A

Tablets containing a dose of 50 mg of active product are prepared according to the usual technique, said tablets having the following composition:

| | |
|---|---|
| riluzole | 50 mg |
| mannitol | 64 mg |
| microcrystalline cellulose | 50 mg |
| polyvidone, excipient | 12 mg |
| sodium carboxymethyl starch | 16 mg |
| talc | 4 mg |
| magnesium stearate | 2 mg |
| anhydrous colloidal silica | 2 mg |

mixture of methylhydroxypropylcellulose
polyethylene glycol 6000, titanium dioxide
(72-3, 5-24, 5)
q.s. 1 film-coated tablet having a final weight
of 245 mg

### EXAMPLE B

Gel capsules containing a dose of 50 mg of active product are prepared according to the usual technique, said gel capsules having the following composition:

| | |
|---|---|
| riluzole | 50 mg |
| cellulose | 18 mg |
| lactose | 55 mg |
| colloidal silica | 1 mg |
| sodium carboxymethyl starch | 10 mg |
| talc | 10 mg |
| magnesium stearate | 1 mg |

### EXAMPLE C

An injectable solution containing 10 mg of active product is prepared, said solution having the following composition:

| | | |
|---|---|---|
| riluzole | 10 | mg |
| benzoic acid | 80 | mg |
| benzyl alcohol | 0.06 | cm³ |
| sodium benzoate | 80 | mg |
| 95% ethanol | 0.4 | cm³ |
| sodium hydroxide | 24 | mg |
| propylene glycol | 1.6 | cm³ |
| water | q.s 4 | cm³ |

### Methods

Purification and culture of peripheral blood mononuclear cells (hereinafter referred to as PBMCs)

The lymphocyte cultures are prepared according to the method described by Achour et al., Antimicrob. Agents, Chemother 42, 2482-2491 (1998).

The PBMCs were isolated from citrate-treated fresh whole blood from donors who were, respectively, healthy and infected with HIV-1 by density-gradient centrifugation using a device known as Ficoll-Hypaque (Eurobio, Les Ullis, France). The harvested cells were resuspended at 10⁶/ml in RPMI 1640 culture medium containing 10% of decomplemented human AB serum, non-essential amino acids, 10 U/ml of penicillin (Sigma), 100 μg/ml of streptomycin (Sigma), 2 mM of L-glutamine (Sigma), 1 mM of sodium pyruvate (Sigma), 10 mM of HEPES buffer, plus 20 IU/ml of interleukin 2 (Boehringer Mannheim, Germany); this medium constitutes the complete culture medium, i.e. abbreviated to CM. The cells were subsequently seeded, at 4×10⁶/well into 6-well culture plates (Nunc, Roskilde, Denmark) and were stimulated with 100 ng/ml of anti-CD3 monoclonal antibody (Pharmingen, Los Angeles, Calif., USA) plus 100 ng/ml of anti-CD28 monoclonal antibody (Pharmingen, Los Angeles, Calif., USA) in the absence or in the presence of various concentrations of riluzole (10⁻⁴/10⁻¹⁰ M). The cultures were maintained at 37° C. in humidified air containing CO₂. The culture media were changed every 4-5 days, the cultures being maintained at a constant viable-cell density of 1×10⁶ cells/ml. At each passage, viable cells were counted by trypan blue staining and the supernatants were harvested for storage at −20° C.

### Apoptosis Test

The apoptotic cells were measured using propidium iodide and FITC-labeled annexin V, which is a phospholipid-binding protein that binds preferentially to the phosphatidylserine exposed at the surface of the cells in the initial phase of apoptosis, by means of a commercially available kit (Immunotech, Marseille, France). The cells that were propidium-iodide-negative and annexin-V-positive were identified as being apoptotic cells, whereas those that were positive for both propidium iodide and annexin V were considered to be pre-necrotic cells. The apoptosis caused by the anti-FAS monoclonal antibody (CD95/APO-1) (Immunotech, Marseille, France) was thus measured since it is well known that Fas/Fas ligand apoptosis is amplified in AIDS disease.

### Flow Cytometry

Counting of T cells having CD4+ and CD8+ phenotypes, of the Ki-67 activation marker and of annexin V was carried out by flow cytometry analysis (FACScan, Becton Dickenson, San José, Calif., USA). A series of monoclonal antibodies directed against the following surface markers of T cells was used: CD4-PerCP, CD8-PE (Becton Dickinson, France), Ki-67-FITC (Pharmingen, Los Angeles, Calif., USA), Annexin-FITC (Immunotech, Marseille, France).

### Viral Quantification Test

The viral production was determined by measuring the HIV RNA in the cell-free supernatants by means of a multiple-primer-induced overlapping amplification test, with a detection threshold of 10 copy equivalents/ml (Lu et al., Nat. Med., 1999). The primers used are the following:

F1/R1 (F1, sense nucleotides 1359-1387 of HIV-1 HXB2 sequence, GenBank accession No. K03455,

SEQ ID No. 1:
5′-GTGGGGGGACATCAA-GCAGCCATGCAAAT-3′

antisense nucleotides 1630-1659 of HXB2,

SEQ ID No. 2:
5′-CCTTTGGTCCTTGTCTTATGTCCAG-AATGC-3′).

The RNA was extracted from 100 μl of the culture supernatants, using a commercial RNA isolation solution (RNAzol; WAK-Chemie Medical, Bad Hamburg, Germany) and then amplified by RT-PCR using primers for HIV-1 gag (Lu et al., Nat. Med., 1999). The amplified products were then detected using a solid-phase technique.

Briefly, 10 μl of the amplified products were detected, denatured for 15 minutes (0.4 M EDTA/2 mM NaOH), and then captured on a microplate coated with streptavidin containing a biotinylated specific probe and the hybridization buffer. The hybridization at 37° C. for 30 minutes was followed by washing with the washing buffer (PBS/0.3% Tween 20). The DNA-DNA hybrid formed was detected by adding a mouse anti-double-stranded DNA monoclonal antibody and then a second antibody directed against the first, obtained in goats, and labeled with alkaline phosphatase. After addition of the substrate (p-nitrophenyl phosphate, pNPP), the optical density was measured at 405/450 nm using a microplate reader (Dynatech MRX). The optical density of the samples is directly correlated to the number of HIV-1 sequences specifically amplified in the sample. The viral load is thus calculated by means of the standard calibration curve which follows a log-log regression mode.

The proviral DNA was determined by means of a modified Muprovama test. To this effect, 2×10⁵ cells were used for purification of cellular DNA with a commercial kit (QIAmp® DNA minikit, Quiagen GmbH, Hilden, Germany). Four standard dilutions (10, 100, 1000 and 10 000 copies) in an equivalent of 10⁵ cells of DNA from PBMCs of a donor negative with respect to HIV, and of plasmid (pBH10-R3) HIV-1 DNA were used as external standard in each experiment. An equivalent of 10⁵ cells of purified sample DNA and 4 DNA standards were used for a specific amplification of HIV by means of the Muprovama test, while an equivalent of 10² cells of each purified sample DNA or of standard DNA had been purified in parallel for the calibration of the genomic DNA with a pair of primers of the β-actin genomic sequence (Genebank genebase accession No. M10277: 2.280-2.301, sense and 2.606-2.538, antisense). After amplification, 10 μl of HIV reaction product or of β-actin reaction product of each sample were distributed into the 96 wells of a microtitration plate coated with streptavidin, preincubated with a biotin-labeled probe specific for HIV-1 or for β-actin. An Elisa assay coupled to hybridization was carried out. The optical signal of each HIV hybrid well was automatically corrected for the difference between the signal of the corresponding β-actin hybrid well and the mean of the β-actin signals of the standard DNA with software (Dynex, Chantilly, USA). The proviral DNA load was calculated using the standard calibration curve (log-log regression model in the range of 1 to 10⁵ copies of HIV-1 DNA per 10⁶ cells) generated on the basis of signals of four dilutions of the HIV-1 DNA external standard.

### Detection of Cytokines by Real-Time PCR

Using real-time PCR, 2 μg of total RNA is first reverse-transcribed by means of a kit (High Capacity cDNA Archive kit PN: 4322171, Applied Biosystems, Foster City, Calif.) using random primers and the MultiScribe RT enzyme (5 U/μl) for 2 hours at 37° C. The method of labeling with a SYBR Green, a fluorescent molecule which intercalates into the minor groove of the double helix, was used. The reaction is carried out using a thermocycler (ABI prism 7900 HT, Applied Biosystems, Foster City, Calif.). Each point studied was composed of 5 μl of SYBR Green PCR Master Mix (PN:4344463; Applied Biosystems, Foster City, Calif.), 0.3 μl of sense and antisense primer at a concentration of 10 μM (Proligo), 3.4 μl of Rnase-free water and 1 μl of cDNA. After a denaturation step at 95° C. for 15 min, the amplification is carried out after 40 cycles at 95° C. for 15 s, 60° C. for 30 s and 72° C. for 30 s. At the end of the PCR, a dissociation step corresponding to the amplification peak, which takes place in a cycle of 95° C. for 15 s, 60° C. for 15 s and 95° C. for 15 s, verifies the validity of the primers. The primers for all the genes studied were chosen according to the Oligo Explorer 1.1 amplification conditions. The samples were studied in duplicate in the presence of a negative control, composed of Master Mix and the primers, but without the cDNA. Glyceraldehyde phosphate dehydrogenase (GAPDH) was used as endogenous control gene.

The effect of the treatments on the levels of mRNA of interest is studied using the method of comparison of CTs (Peinnequin et al., 2004) or number of cycles necessary to reach a threshold fluorescence value (software SDS 2.1, AB prism).

The sequences of the primers used are the following (Proligo LLC, Boulder, Colo., USA) (forward-reverse):

SEQ ID No. 3 and SEQ ID No. 15:
GAPDH
(5′-AACAGCCTCAAGATCAGCAA-3′)-
(5′-CAGTCTGGGTGGCAGTGAT-3′)
SEQ ID No. 4 and SEQ ID No. 16:
TLR-2
(5′-CTCTCGGTGTCGGAATGTC-3′)-
(5′-AGGGGGGATTGAAGTTCTC-3′)
SEQ ID No. 5 and SEQ ID No. 17:
TLR-3
(5′ACGAGACCCATACCAACATCC-3′)-
(5′-TTCCCAGACCCAATCCTTATC-3′)
SEQ ID No. 6 and SEQ ID No. 18:
TLR-7
(5′-GACCTCAGCCACAACCAAC-3′)-
(5′-TAACCCACCAGACAAACCAC-3′)
SEQ ID No. 7 and SEQ ID No. 19:
TLR-9
(5′-CTACAACCGCATCGTCAAAC-3′)-
(5′-CATTCAGCCAGGAGAGAGAAC-3′)
SEQ ID No. 8 and SEQ ID No. 20:
IFN-α
(5′-ACTTTGGATTTCCCCAGGA-3′)-
(5′-CAGGCACAAGGGCTGTATT-3′)
SEQ ID No. 9 and SEQ ID No. 21:
IFN-β
(5′-ATCTAGCACTGGCTGGAATGAG-3′)-
(5′-TTCGGAGGTAACCTGTAAGTCTG-3′)
SEQ ID No. 10 and SEQ ID No. 22:
IFN-γ
(5′-GGGTTCTCTTGGCTGTTACTG-3′)-
(5′-GCATCTGACTCCTTTTTCGC-3′)
SEQ ID No. 11 and SEQ ID No. 23:
IL-10
(5′-GCTGGAGGACTTTAAGGGTTACCT-3′)-
(5′-CTTGATGTCTGGGTCTTGGTTCT-3′)
SEQ ID No. 12 and SEQ ID No. 24:
IL-12P40
(5′-TGGAGTGCCAGGAGGACAGT-3′)-
(5′-TCTTGGGTGGGTCAGGTTTG-3′)
SEQ ID No. 13 and SEQ ID No. 25:
IL-15
(5′-CCATCCAGTGCTACTTGTGTTTAC-3′)-
(5′-CCAGTTGGCTTCTGTTTTAGGAA-3′)
SEQ ID No. 14 and SEQ ID No. 26:
IL-18
(5′-GACGCATGCCCTCAATCC-3′)-
(5′-CTAGAGCGCAATGGTGCAATC-3′).

### Measurement of the Functional Activity of Interferon

The functional test is evaluated by means of the ability thereof to inhibit the cytolytic capacity of the vesicular stomatitis virus (VSV) with respect to MDBK cells.

### Measurement of Interleukin-15 Production

The IL-15 production in the supernatant of cells conditioned with riluzole is estimated using the ELISA enzymatic immuno assay (R&D, Quantikine, UK).

### Measurement of the Inhibition of Tumor Cell Proliferation

The TG 180 line (Crocker sarcoma tumor) is cultured in the presence of various concentrations of Riluzole. After 5 days, the proliferation of the cells is estimated by tritiated (3H) thymidine incorporation. Other tumor lines were used, namely the H9 line (T lymphoma), the NB4 line (line derived from an acute promyelocytic leukemia) and the Jurkat line (T-lymphoma-derived lymphoblastic T cell line).

### Results

### Increased Survival of T Cells Under the Effect of Riluzole:

The percentage cell survival on the day the cultures are passaged is estimated in the following way:

$$\frac{Number of viable cells on the day - number of cells on day 0}{Number of cells on day 0} \times 100 \%$$

The effect of riluzole was first studied on the lymphocytes of 2 patients suffering from AIDS.

The patient PE had received no therapy and had the following parameters (T4:467/mm³, T8:1662/mm³, viral load: 60824 copies/ml of plasma). The cell survival, on day 13 of the culture, was increased by 216% compared with the control cells, at the dose of 10⁻⁶ M and by 378% at the dose of 10⁻⁷ M (FIG. 1A).

The patient PE was receiving a therapy and had the following parameters (T4:880/mm³, T8:1280/mm³, viral load: 250 copies/ml of plasma). The cell survival, on day 13 of the culture, was increased by 169% compared with the control cells, at the dose of 10⁻⁶ M and by 202% at the dose of 10⁻⁷ M (FIG. 2A).

The dose effect of riluzole was then studied on the lymphocytes of an AIDS patient AR receiving no therapy and having the following parameters (CD4:291/mm³ (17%), CD8:872/mm³ (51%), virus:43576 copies/ml of plasma). The cell survival on day 13 of the culture, was increased by 670% compared with the control cells, at the dose of 10⁻⁸ M, by 407% at the dose of 10⁻⁷ M and by 230% at the dose of 10⁻⁸ M (FIG. 3A). As a control, the dose effect of riluzole was also studied on the lymphocytes of a seronegative healthy individual. The cell survival, on day 13 of the culture, was increased by 271% compared with the control cells, at the dose of 10⁻⁸ M, by 228% at the dose of 10⁻⁷ M and by 157% at the dose of 10⁻⁶ M (FIG. 4A).

### Effect of Riluzole on Cell Death in Lymphocytes in Culture

Furthermore, it can be noted in FIGS. 1B, 2B and 3B hereinafter that, for example, in the case of the cells treated with riluzole, the percentage of dead cells in the culture is maintained at around 2 to 10%, whereas it varies from 10 to 25%, from the 3rd day of the culture. As controls, the lymphocytes of a healthy individual, cultured in the presence or absence of riluzole, do not show a specific effect on mortality (FIG. 4B).

The presence of riluzole in the culture medium therefore protects the mononuclear cells against HIV-1-induced cell death and enables proliferation of lymphocytes.

### Negative Regulation of Apoptosis and of Ki-67 Expression in Lymphocytes by Riluzole

T cell apoptosis and the expression of Ki-67 (which is a marker for T cell proliferation) are also regulated after 13 days in the presence of riluzole. Specifically, in the concentration window for the drug (10⁻⁷-10⁻⁸ M) riluzole inhibits the total apoptosis expressed by all the lymphocytes by 66%, the apoptosis of CD8 lymphocytes by 70% and the apoptosis of CD4 lymphocytes by 80% (FIGS. 5A, 5B, 5C), while the apoptosis that is expressed weakly by the lymphocytes of a healthy donor is not changed by the treatment with riluzole (FIG. 4C).

The study of the expression of the Ki-67 antigen (which is a marker for proliferation-competent T cells) shows that it is negatively regulated. This effect is characterized by an inhibition of the order of 33% by all of the lymphocytes, of 37% by the CD8 lymphocytes and of 50% by the CD4 lymphocytes (FIGS. 6A, 6B, 6C). These results reflect the recovery of T cells after completion of their cell proliferation cycle.

### Negative Regulation of FAS/FAS Ligand Apoptosis by Riluzole:

As indicated above, it is well established that the lymphocytes of HIV-1-infected patients are sensitive to apoptosis induced by the expression of FAS (CD95/APO-1) and of its agonist, the anti-FAS antibody. Thus, this antibody was made to act on the lymphocytes of the patent AR in the absence or in the presence of riluzole and apoptosis was measured through the expression of annexin V. The results in FIG. 7 indicate that the apoptosis of control cells coupled to the antibody reaches the threshold of 50% expression, whereas the cells conditioned with riluzole at the dose of 10⁻⁸ M express it only at 15% (62% inhibition). This percentage inhibition is comparable for the cells cultured without anti-FAS antibody.

### Effect of Riluzole on HIV-1 Replication:

Having thus observed the restoring of the proliferative activity of the T cells of infected patients treated or not treated with antivirals (tritherapy; antipolymerase plus antiprotease) and the inhibition of apoptosis and of cell death, the expression of the virus in the cultures was examined.

The concentrations of HIV-1 RNA in the supernatants collected from the cultures of T cells of patients were different in the presence or in the absence of the compound. The HIV-1 RNA in the supernatants of the cultures, originating from the untreated patient PE having a high viral load, conditioned with riluzole (10⁻⁷ M), was increased at day 13 of the culture by 100%; the viral load being high from the start, and the virus egress peak for the control occurring at day 6. At days 3 and 6 of the culture, the virus measurement is virtually the same; however, at days 10 and 13, while the curve for the controls shows the beginnings of a decrease, that for the conditioned cultures (10⁻⁷ M) remains twice as high (FIG. 1C). On the other hand, for the cells originating from the patient PR subjected to tritherapy and having a viral load at the limit of detectability, from the 3rd day of culture, a highly substantial virus egress is observed on the cells conditioned with riluzole (10⁻⁷ M) compared with the control (1.1 log₁₀ more) (FIG. 2C). The virus egress peak occurs at day 10 of the control culture due to the history of the lymphocytes. At day 13 of the culture, although the viral expression of the control cells stagnates, that of the treated cells peaks at a high threshold (0.7 log₁₀ more).

The dose effect of riluzole was then studied on the expression of the viral RNA of the lymphocytes of the AIDS patient AR receiving no therapy and having a plasma viral load of 43576 copies/ml. The virus egress peak occurs at day 10 of the culture. In comparative terms, the cells conditioned with riluzole, from the 6th day of the culture, expressed 0.8 logₙ more viral RNA than the control at the doses of 10⁻⁸ M, 10⁻⁷ M and 10⁻⁸ M. This production continued to increase at day 13 (up to 0.7 log₁₀ more) (FIG. 3C).

### Effect of Riluzole on the Integrated Virus (Proviral DNA):

The results obtained are given in table 1 below:

TABLE 1
The effect of riluzole (0.1 μM) on the proviral DNA of
lymphocytes of untreated and treated HIV-1 patients,
cultured in the absence or in the presence of riluzole

| | Day 3 of culture | | Day 6 of culture | |
|---|---|---|---|---|
| | (−) | (+) | (−) | (+) |
| ^{#}TT(−), | 6.8 ± 0.1 | 3.7 ± 0.2** | 5.7 ± 0.3 | 0.7 ± 0.1** |
| V: 68.000* | | | | |
| ^{#}TT(+), | 4.5 ± 0.3 | 3 ± 0.2** | 3.8 ± 0.2 | 0.2 ± 0.1** |
| V: 250* | | | | |
| ^{#}TT(+), | 5.3 ± 0.2 | 2 ± 0.3** | 4.8 ± 0.3 | 0.3 ± 0.1** |
| V: 44.000* | | | | |

^{#}TT(−): Patients without tritherapy, TT(+): Patients receiving tritherapy
*Number of copies per ml of blood. Log of proviral HIV DNA (copies [cp]/10⁶ cells). Data expressed in the form of mean + S.D. (standard deviation) of the measurements.
**Value P (Wilcoxon) < 0.01 by comparison with the culture medium alone.

The data in table 1 demonstrate that riluzole is capable of specifically decreasing the expression of the integrated virus (p<0.01) of cells originating from AIDS patients.

These results show that riluzole, while inhibiting virus-induced apoptosis, thus promoting lymphocyte proliferation, is at the same time capable of acting on the “reservoir” cells and of pushing them to bring out the weakly replicative virus.

### Effect of Riluzole on the Expression of Cytokine Genes:

In order to associate the described effects of riluzole with the expression of cytokines, the inventors measured the expression of cytokine genes by real-time PCR. The results obtained showed that riluzole is capable of inducing the expression of the IL-15, interferon-α and interferon-β genes and the Toll receptor 3 gene (FIG. 8).

### Effect of Riluzole on the Production of Interferon and of IL-15:

The activation of the interferon and IL-15 genes is associated with the production of these two cytokines in the supernatant of cells treated with riluzole. The functional activity of interferon is determined by virtue of its ability to inhibit the cytolytic capacity of VSV. The production of IL-15 is, for its part, estimated by means of a commercial ELISA assay. Thus, cells treated with 10⁻⁸ M of riluzole are capable of producing 6 pg/ml of IL-15. The results obtained are given in table 2 below:

TABLE 2
Effect of riluzole (10⁻⁸ M) on the production of IL-15
and of interferon α/β

| | |
|---|---|
| Interleukin 15 | Interferon α/β |

| (−) Riluzole | (+) Riluzole | (−) Riluzole | (+) Riluzole |
|---|---|---|---|
| 1* | 15* | 20** | 32 000** |

*The measurement of IL-15 production is estimated by the Elisa technique (R&D kit, UK); the values correspond to picograms per ml (pg/ml) of culture.
**The interferon values correspond to the number of international units per ml of culture.

When the cells are conditioned with riluzole at the concentration of 10⁻⁸ M, they become capable of producing interferon α/β capable of inhibiting the cytolytic capacity of VSV. The amount thus produced is estimated at 32 000 international units (table 2).

### Effect of Riluzole on Tumor Cell Proliferation:

When tumor cells of the ATG 180 line (Brocker sarcoma) are cultured with various concentrations of riluzole (10⁻⁴-10⁻⁸ M), inhibition of the proliferation of the cells is observed compared with the controls. At 10⁻⁴ M, an 86% inhibition of proliferation is observed, it is 40% at 10⁻⁵ M and 30% at 10⁻⁵ M (FIG. 9a). When other tumor cells are used, such as the Jurkat line (FIG. 9b), the NB4 line (FIG. 9c) and the H9 line (FIG. 9d), inhibition of the proliferation of these tumor cells is also observed.

Inhibition of viral RNA by addition of riluzole to an antiviral compound such as AZT

Cells originating from the patients PE, PR and AR (see FIGS. 1-3) are cultured from day 12 in the presence of riluzole and in the presence of AZT.

After 6 days of culture, the presence of the virus is measured by measuring the viral RNA, the virus integrated into the cell in the form of proviral DNA, the cell survival rate and the cell viability.

The results are given in table 3 below (in this table, Ril=riluzole).

An inhibition of viral RNA is observed with AZT at the doses of 10 μg/ml and of 1 mg/ml concurrently with riluzole concentrations ranging from 10⁻⁵ to 10⁻⁸ M.

As shown by the results in the table, AZT at 10 μg/ml, which exerts an antiviral effect, also exhibits a suppressor (antiproliferative) effect on the control cells.

The combined use of riluzole and AZT makes it possible to maintain the proliferation and demonstrates the restoring effect of riluzole with respect to the activity of a drug known for its antiproliferative effect at the dose of 10 μg/ml.

When the AZT is used at 1 μg/ml, the antiviral effect is partial, or even equivalent, on the control cells.

On the other hand, the concomitant addition of riluzole at the concentrations indicated makes it possible to sustain both viral RNA inhibition and the maintenance of cell proliferation. It should also be noted that riluzole (10⁻⁶/⁻⁹ M) makes it possible to significantly decrease the level of integrated virus (proviral DNA) and of the released virus (viral RNA) to a level at which AZT (1 μg/ml) shows no viral inhibition, while at the same time conserving its cell-survival-restoring properties.

TABLE 3

| | | ⁽²⁾Proviral | | |
|---|---|---|---|---|
| | ⁽¹⁾Viral | DNA | ⁽³⁾Cell | |
| | RNA | Number of | survival | ⁽⁴⁾Viability |
| | Number of | copies/ | rate | % |
| Conditioning | copies/ml | 10⁶ cells | (×100%) | dead cells |

Individual PE

| | | | | |
|---|---|---|---|---|
| −AZT | 25 000 | 5.3 | 5 | 35 |
| +AZT 10 μg/ml | <100 | 5 | 3 | 20 |
| //+Ril 10⁻⁶ M | <100 | 0.1 | 12 | 7 |
| //+Ril 10⁻⁷ M | <100 | 0.1 | 15 | 6 |
| +AZT 1 μg/ml | 12 000 | 6.5 | 3 | 29 |
| //+Ril 10⁻⁶ M | <100 | 0.1 | 14 | 8 |
| //+Ril 10⁻⁷ M | <100 | 0.2 | 18 | 16 |

Individual PR

| | | | | |
|---|---|---|---|---|
| −AZT | 10 000 | 4.5 | 15 | 25 |
| +AZT 10 μg/ml | <100 | 4.8 | 13 | 20 |
| //+Ril 10⁻⁶ M | <100 | 0.2 | 45 | 7 |
| //+Ril 10⁻⁷ M | <100 | 0.1 | 50 | 6 |
| +AZT 1 μg/ml | 7000 | 4.9 | 12 | 19 |
| //+Ril 10⁻⁶ M | <100 | 0.2 | 53 | 8 |
| //+Ril 10⁻⁷ M | <100 | 0.1 | 48 | 9 |

Individual AR

| | | | | |
|---|---|---|---|---|
| −AZT | 60 000 | 5 | 7 | 20 |
| +AZT 10 μg/ml | <100 | 5.4 | 4 | 25 |
| //+Ril 10⁻⁶ M | <100 | 0.1 | 15 | 11 |
| //+Ril 10⁻⁷ M | <100 | 0.08 | 19 | 9 |
| //+Ril 10⁻⁸ M | <100 | 0.15 | 27 | 10 |
| //+Ril 10⁻⁹ M | <100 | 0.2 | 12 | 9 |
| //+Ril 10⁻¹⁰ M | <100 | 3 | 6 | 16 |
| +AZT 1 μg/ml | 35 000 | 6.1 | 5 | 28 |
| //+Ril 10⁻⁶ M | <100 | 0.1 | 17 | 10 |
| //+Ril 10⁻⁷ M | <100 | 0.1 | 20 | 9 |
| //+Ril 10⁻⁸ M | <100 | 0.15 | 30 | 8 |
| //+Ril 10⁻⁹ M | 17 000 | 0.4 | 16 | 10 |
| //+Ril 10⁻¹⁰ M | 23 000 | 4 | 8 | 19 |

⁽¹⁾Number of copies of viral RNA per ml of culture medium by means of RT PCR viral quantification test.
⁽²⁾Log of proviral HIV DNA (copies [cp]/10⁶ cells).
⁽³⁾Cell survival is estimated by virtue of the rate (number of cells day 18 − number of cells day 12) × 100/number of cells day 12.
⁽⁴⁾The cell viability is estimated by counting with trypan blue.

1. A method of amplifying and restoring the immune function of a person in need of said amplifying and restoring as a result of treatment of infectious disease or cancer said method comprising administering riluzole or of pharmaceutically acceptable salts of riluzole to said person such that said immune function is amplified and restored.

2. The method as claimed in claim 1, wherein said riluzole or pharmaceutically acceptable salts are administered in forms for oral, parenteral, for example intravenous, or topical administration, or else rectal administration, in particular in the form of suppositories.

3. The method as claimed in claim 1 wherein said administering induces interferon α/β and of interleukin 15 and said person suffers from a dysfunction associated with a pathological condition of exogenous or endogenous origin.

4. The method as claimed in claim 3, characterized in that the pathological condition is associated with an attack of iatrogenic origin, induced by the action of immunosuppressants such as cyclosporine or by the action of corticosteroids.

5. The method as claimed in claim 1 wherein said infectious disease is a pathological condition associated with a viral attack.

6. The method as claimed in claim 5, wherein said infectious disease is of retroviral origin due to HIV type I or II and HTLV I and II, or infections with herpes viruses.

7. The method as claimed in claim 5, wherein the infectious disease is of viral origin due to hepatitis viruses.

8. The method as claimed in claim 1 wherein the treatment comprises administration of the combination with one or more compounds having antiviral properties.

9. The method as claimed in claim 8, wherein the antiviral compound is chosen from DDI, DDC, antiproteases, 3TC and AZT.

10. The method as claimed in claim 1 wherein the infectious disease is associated with a parasitic attack, such as leishmaniasis, malaria or schistosomiasis.

11. The method as claimed in claim 1 wherein the cancer is of tumoral origin, such as an immunological tumor, for instance myeloma and leukemias, or a tissue tumor.

12. The method as claimed in claim 2, wherein said person is being treated for a pathological condition associated with an autoimmune attack.

13. The method as claimed in claim 11, characterized in that said riluzole or a pharmaceutically acceptable salt of riluzole is medicaments are administered at a rate of 50 to 200 mg/day of at least one compound of formula (I), as active ingredient, at unit doses of 25 to 100 mg.
